(19) United States
(12) Patent Application Publication
(10) Pub. No.: US 2006/0120945 A1
(43) Pub. Date: Jun. 8, 2006

(54) Reusable apparatus for gas generation

(75) Inventors: John J. Warner, Manchester-By-The-Sea, MA (US); Gary A. O'Neill, Tyngsborough, MA (US); Richard A. Hamilton, Beverly, MA (US)

Correspondence Address: LAHIVE & COCKFIELD, 28 STATE STREET, BOSTON, MA, 02109, US

(73) Assignee: SELECTIVE MICRO TECHNOLOGIES, LLC, Beverly, MA (US)

(21) Appl. No.: 11/302,602

(22) Filed: Dec. 14, 2005

Related U.S. Application Data
(63) Continuation of application No. PCT/US04/19679, filed on Jun. 16, 2004.
(60) Provisional application No. 60/479,029, filed on Jun. 16, 2003.

(51) Int. Cl. A61L 2/20

(52) U.S. Cl. 423/477; 422/305; 422/37

(57) ABSTRACT

Disclosed herein are reusable apparatus for the generation of a gas including a reactant chamber defined at least in part by a gas permeable material and a resealable opening. In certain embodiments the gas permeable material is substantially impervious to the passage of liquid and/or allows for the controlled passage of gas. Also disclosed are methods for using the apparatus, reusable reactant chambers, kits including reactants and reactant refill kits.

### RELATED APPLICATIONS

This application is a continuation of International Application No. PCT/US2004/019679, filed Jun. 16, 2004, which claims priority to U.S. Provisional Application No. 60/479,029, filed Jun. 16, 2003. The contents of each of these references are hereby incorporated by reference herein.

### TECHNICAL FIELD

The invention relates generally to apparatus for gas generation. More specifically, the invention relates to reusable apparatus having a resealable opening and for use in gas generation.

### BACKGROUND

The use of gas for retarding, controlling, killing or preventing microbiological contamination (e.g., bacteria, fungi, viruses, mold spores, algae and protozoa); retarding, preventing, or controlling biochemical decomposition; controlling respiration, deodorizing and/or retarding and preventing chemotaxis to name a few, is known. Such gases include, but are not limited to, chlorine dioxide, sulfur dioxide, nitrogen dioxide, nitric oxide, nitrous oxide, carbon dioxide, hydrogen sulfide, hydrocyanic acid, and dichlorine monoxide.

Although it has great beneficial characteristics, chlorine dioxide can not be transported commercially as a concentrated gas for its use and instead must be generated at the site where it is used. Transportable devices for generating gas generally operate inefficiently and produce unsatisfactory levels of byproducts or reactants that can remain as a residue and contaminate the surrounding environment. For example, in the case of chlorine dioxide gas generation, the byproduct chlorite leaves residue on food handling equipment and medical and dental surfaces. Human contact with such residue should be avoided or substantially minimized according to FDA and EPA regulations. Another disadvantage of current techniques is the inability to reuse existing devices, resulting in added expense and waste.

### SUMMARY OF THE INVENTION

The present invention provides a reusable apparatus for the controlled, on-site generation of gases, such as sulfur dioxide and chlorine dioxide. Employing the apparatus of the invention, gas can be produced safely, efficiently and economically. Moreover, because the apparatus can be reused, waste can be minimized. In preferred embodiments, the apparatus can minimize or even eliminate the release of any of the reactants or byproducts from the apparatus.

In one aspect, the present invention provides a reusable apparatus for the generation of a gas, the apparatus including a reactant chamber defined at least in part by a gas permeable material and a resealable opening. The gas permeable material may be substantially impervious to the passage of liquid and/or may allow for the controlled passage of gas. In a particular embodiment, the gas permeable material is formed of a robust material capable of providing structural support. In one embodiment, the reactant chamber further includes a gas impermeable material. In another embodiment, the resealable opening is adapted for closure with a cap, a lid, a clamp, or a zip-lock mechanism.

In yet another embodiment, the apparatus includes a supporting element disposed adjacent to at least a portion of the chamber. The chamber can include two or more compartments.

In certain embodiments, the apparatus further includes one or more reactants disposed in the chamber. The reactants may be mixed. In addition, in various embodiments, at least one of the reactants is disposed in a sachet; at least one of the reactants is disposed in a frangible device; at least one of the reactants is a tablet; at least one of the reactants is in the form of a gel, a liquid or a powder; and/or at least one of the reactants is in the form of a pellet, a ring, or a disc.

In other embodiments, the apparatus includes a hydrotalcite (e.g., a thermally treated hydrotalcite) disposed in the chamber. In addition, the apparatus may further include a relief valve.

In another aspect, the present invention provides a device for gas generation including a reservoir and any of the reusable apparatus disclosed herein. The reservoir may be in fluid communication with the reactant chamber. In various embodiments, the reservoir may be a humidifier, a spray bottle, a tank, a cartridge, a bottle, a drum, a storage drum, an air conditioner reservoir, or a conduit.

In another aspect, the present invention provides a reusable reactant chamber adapted to receive one or more reactants for the generation of gas, defined at least in part by a gas permeable material and a resealable opening.

In yet another aspect, the present invention provides a method of generating gas (e.g., chlorine dioxide) by providing a reusable apparatus including a reactant chamber defined at least in part by a gas permeable material and a resealable opening, where one or more reactants are disposed in the chamber; and exposing the one or more reactants to an initiating agent (e.g., water vapor, water or a combination thereof) so as to generate gas. The apparatus may have one or more of the features described above.

In various embodiments, at least one of the reactants and/or the initiating agent is present in a frangible device. According to the methods disclosed herein the frangible device may be punctured so as to expose the one or more reactants and/or the initiating agent. In other embodiments, the apparatus is exposed to an environment comprising the initiating agent, thereby exposing the one or more reactants to the initiating agent. The apparatus may be submerged in the initiating agent. Alternatively, the apparatus may be partially submerged in the initiating agent.

In another aspect, the present invention provides a kit including a reusable reactant chamber defined at least in part by a gas permeable material; and one or more reactants.

In yet another aspect, the present invention provides a refill kit including one or more reactants for use in a reusable reactant chamber and optionally, a means for delivery of the one or more reactants to a reusable reactant chamber.

### BRIEF DESCRIPTION OF THE DRAWINGS

The invention is pointed out with particularity in the appended claims. The drawings are not necessarily to scale, emphasis instead generally being placed upon illustrating the principles of the invention. The advantages of the invention described above, as well as further advantages of the invention, can be better understood by reference to the description taken in conjunction with the accompanying drawings, in which:

FIGS. 1A-C are perspective views of kits including exemplary reusable apparatus of the present invention, and a plurality of reactants. FIG. 1A features a zip-lock resealable opening. FIGS. 1B and 1C feature reusable apparatus having threaded resealable openings and supporting elements disposed outside and inside the gas permeable material, respectively.

FIGS. 1D-G are side views of exemplary reusable apparatus of the present invention, each featuring a reactant chamber (FIGS. 1D-E), and a reservoir adapted to receive the reusable reactant chamber (FIGS. 1F-G).

FIG. 1H is a side view of an exemplary reusable apparatus of the present invention, featuring multiple layers of gas permeable material.

FIG. 1I is a side view of an exemplary reusable apparatus of the present invention, formed at least, in part, of a robust gas permeable material.

FIG. 2A is a side view of an exemplary reusable apparatus of the present invention featuring a reactant chamber having a threaded resealable opening, the mated lid of which is adapted to couple to a reservoir.

FIG. 2B is a side view of an exemplary apparatus of the invention featuring a reactant chamber having a threaded resealable opening, the mated lid of which is adapted to couple to a reservoir. The reactant chamber is partially submerged into liquid contained in a reservoir.

FIG. 2C is a side view of an exemplary reusable apparatus of the present invention featuring a reactant chamber having a threaded resealable opening that is also adapted to couple to a reservoir. As shown, the reactant chamber is submerged into the liquid contained in the reservoir.

FIGS. 3A-3B are side views of exemplary apparatus of the invention featuring reactant chambers plumbed into conduits.

FIG. 4 is a side view of an exemplary apparatus of the invention featuring a reactant chamber having a supporting element and a reservoir.

FIG. 5 is a side view of an exemplary apparatus of the invention featuring a reactant chamber and a supporting element that can be introduced into or connected to a conduit.

FIGS. 6A and 6B are graphs of chlorine dioxide generation by reusable apparatus constructed as described in Example 1.

FIG. 7 is a graph of chlorine dioxide generation by a reusable apparatus constructed as described in Example 2.

### DETAILED DESCRIPTION OF THE INVENTION

The present invention is generally directed to reusable apparatus for the generation of gas. The invention further contemplates kits that include the reusable apparatus and/or reactant in a variety of forms. Moreover the invention provides for devices for gas generation, including a reservoir (e.g., a humidifier or a tank) and the reusable apparatus. The present invention also contemplates methods for use of the apparatus and/or kits, including refill kits, for the generation of gas.

Generally, the present invention relates to apparatus and methods for delivering biocidal-effective amounts of a gas such as chlorine dioxide. In certain embodiments, the apparatus and methods of the present invention achieve delivery of a desired amount of gas, at a desired rate, over a desired time period. This is accomplished, at least in part, by disposing suitable reactants in a defined and confined volume such that upon initiation, the reactants, initiating agent, products, and byproducts are held within a desired concentration range. The amount, rate and duration of delivery can be manipulated by, e.g., choice of reactant amount, reactant ratio, gas permeable materials, and reactant chamber volume and construction. Such manipulations can be exercised by the artisan using only routine experimentation in view of the teachings disclosed herein together with knowledge in the art.

One advantage of the present invention is that exemplary embodiments can be employed to conserve resources and cost by reusing the reactant chamber a plurality of times. For example, a resealable opening allows for replacement of reactant as desired. Moreover, the resealable opening allows for removal of any contents remaining after reaction, such as sachets or byproducts. Another advantage of particular embodiments is the improved economy of space and packaging materials as refill kits can provide reactants in loose or premeasured form, without the need for delivery, storage and use of new reactant chambers containing reactant after every use. The use of premeasured forms of reactants further allows for the convenient generation of a desired concentration or amount of gas.

In order to more clearly and concisely describe the subject matter of the claims, the following definitions are intended to provide guidance as to the meaning of specific terms used in the following written description, examples and appended claims. Moreover, the following patents and patent publications disclose a variety of apparatus, systems, devices, reactants and methods, the entire contents of which are expressly incorporated herein by reference: U.S. Pat. No. 6,602,466 of Hamilton et al., issued Aug. 5, 2003, U.S. Pat. No. 6,607,696 of Hamilton et al., issued Aug. 19, 2003, and U.S. Publication No. 20030053931 A1 of Hamilton et al., published Mar. 20, 2003.

A “reactant chamber” is a defined volume within which reactants can be disposed. The reactant chamber is defined at least in part by a gas permeable material, and is closed in the sense that the reactants are substantially retained within the reactant chamber.

A “gas permeable material” is a material, typically a planar material such as a sheet or film, including, but not limited to, perforated films, non-perforated films and membranes, that allows for the passage of gas. Suitable gas permeable materials are identified herein.

A “robust material” is a gas permeable material that has inherent strength and provides structural support. Suitable robust material for use within the invention includes, but is not limited to, polypropylene hydrophobic tube membrane with a pore size of approximately 0.2 micron sold under the designation ACCUREL® PP V8/2 BP by Membrana GmbH (Wuppertal, Germany).

A “resealable opening” is an opening that is capable of being sealed, opened, and resealed at least one time, and preferably multiple times. The terms “seal,” “sealing,” “sealed,” etc., as used herein, refer to a closure of the opening to prevent leakage in and/or out of the reactant chamber, e.g., to prevent leakage of reactants or gas through the opening, or to prevent leakage of liquid in through the opening.

The term “sachet” refers to a closed receptacle for reactant. The sachet is closed in the sense that the reactants, prior to initiation, are substantially retained within the sachet. Sachets can be constructed of, e.g., gas permeable, dissolvable and/or liquid permeable materials. Sachets can be frangible to allow for puncturing of the sachets, resulting in the exposure of the reactants contained therein.

“Impermeable material,” as used herein, refers to a material that substantially prevents or hinders passage of solids, gases and liquids. As contemplated herein, the impermeable materials do not participate in the generation of gas in that, e.g., they do not facilitate contact between initiating agent and reactant. Impermeable materials can be constructed from various materials, including, but not limited to, polymeric material, glass, metal, metallized polymeric material and/or coated papers. Suitable materials for impermeable materials are described in greater detail below. As used herein, “barrier” materials are impermeable materials.

Relying upon the teaching disclosed herein, and the general knowledge in the art, the practitioner of ordinary skill will require only routine experimentation to identify one or more gas permeable materials, robust materials, impermeable materials, sachet materials and/or construct one or more sachets and/or reactant chambers adapted for the purpose at hand.

“Reactant” refers to a reactant or a mixture of reactants that generate gas in the presence of an initiating agent. For purposes of the present invention, initiating agent includes, but is not limited to, gaseous or liquid water. For example, for dry biocidal applications of the present invention, such as for the reduction of molds when shipping fruit, moisture in the atmosphere can be used as an initiating agent. The term “dry application” for the purposes of this application means at least an application where the apparatus of the present invention is not immersed in water or any other liquid. The term “wet application” for the purposes of the present invention means at least an application where the apparatus of the present invention is immersed in water, or other liquid, which can optionally include water. For wet biocidal applications, i.e., when the apparatus of the present invention is immersed in water or any other aqueous medium, such as that used for disinfecting dental or food equipment, the water in which the apparatus is immersed can be used as the initiating agent. Alternatively, the initiating agent can be included within the apparatus, e.g., contained in a frangible pouch disposed within the apparatus.

In a particular embodiment, gas is allowed to enter both the liquid in a reservoir and the enclosed spaces above the reservoir. For example, the gas generating apparatus may be partially submerged in the initiating agent (e.g., water), thereby allowing for initiation of gas generation and escape of the gas into both the water and the space above it. Such techniques are suitable for a variety of applications in accordance with the invention, including decontamination, for example, of water storage tanks.

The term “water vapor selective” as used herein refers to a material that selectively allows permeation of water vapor and substantially impedes permeation of liquid water. More preferably, the material excludes permeation of liquid water. Typically, the water vapor selective material is hydrophobic. The skilled practitioner typically refers to water vapor selective material as water impermeable, although water vapor can permeate the material. By contrast, the skilled practitioner refers to materials that allow permeation of liquid water as water permeable. Suitable water vapor selective materials can be made from a variety of materials including, but not limited to, polytetrafluoroethylene (PTFE), polypropylene (PP), polyethylene (PE), and fluorinated ethylene propylene (FEP).

In one aspect, the present invention is generally directed to reusable apparatus for the generation of a gas, the apparatus including a reactant chamber defined at least in part by a gas permeable material and a resealable opening.

Gas Permeable Materials

The gas permeable materials of the present invention allow for the passage of gas through the material. The materials can be films, sheets, composites, and the like. In one embodiment, the gas permeable material is substantially impervious to liquid. This can be advantageous, e.g., to prevent ingress of liquid initiating agents, such as liquid water, and/or egress of solubilized reactants. It can also be advantageous to retain liquid water or solubilized reactants and byproducts within the reactant chamber such that the reaction efficiency is increased. Additionally or alternatively, the gas permeable materials allow for the controlled passage of gas. This can be advantageous because the gas permeable materials can be used to control the rate and efficiency of the reaction. This allows for control of the duration of the reaction, the amount of gas generated, the reduction of byproducts, and the like.

Gas permeable materials can include robust materials, water vapor selective materials, perforated materials, membranes, selective transmission materials, composite materials, hydrophilic and/or hydrophobic materials, spun bond materials, expanded polymer structure materials (such as ePTFE sold under the BHA-TEX® tradename by BHA Technologies (Kansas City, Mo.)), microporous membrane (such as polyethersulfone membrane sold under the Membrana ACCUREL® Micro PES tradename by Membrana GmbH (Wuppertal, Germany)), woven materials, and non-woven materials.

In particular embodiments, the gas permeable membrane may be or include a robust material. The robust material may be used, for example, when the device may encounter abrasive or rough handling and/or when no support or protective enclosure is practical or desired. In various embodiments, the robust material is bound to, adhered to, spun to or otherwise affixed to the gas permeable membrane by techniques well known in the art. For example, the robust material may be a support layer, e.g., that is bound or otherwise fixed or held to the gas permeable membrane, but which does not interfere with the permeability of the layer. Alternatively or additionally, the robust material may be a web, e.g., to which the gas permeable layer is bound to, adhered to, spun to or otherwise fixed or held. By way of example, the gas permeable membrane may include a copolymer formed, in whole or in part, of a robust material.

Additionally or alternatively, the gas permeable membrane is made of a robust material, in whole or in part. In such embodiments, the nature of the robust material itself (e.g., a cross linked material and/or a material with filler) imparts structural integrity or flexibility without the need for additional supporting elements, although, such supporting elements may be incorporated. Moreover, particular features of the material (e.g., the thickness of the layer) may impart desired structural integrity. Suitable water vapor selective material includes, but is not limited to, an approximately 1,550 micron thick polypropylene hydrophobic tube membrane with a pore size of approximately 0.2 micron sold under the designation ACCUREL® PP V8/2 BP by Membrana GmbH (Wuppertal, Germany).

A preferred gas permeable material is a water vapor selective material. Water vapor selective material is particularly advantageous because it substantially impedes or excludes the diffusion of water soluble species, such as water soluble reactants, additives, and reaction byproducts, out of the apparatus. Materials that allow liquid water permeation allow soluble species such as soluble reactants to permeate the apparatus and enter the environment. Prior attempts have been made to ameliorate or avoid escape of soluble species by using insoluble reactants or by binding or otherwise disposing the soluble species on insoluble materials such as clays and molecular sieves, thereby introducing additional steps and/or expense to the preparation of the apparatus. The water vapor selective materials of the present invention are advantageous because they impede or preclude the permeation of the soluble reactants, additives, and byproducts, thus obviating the need to include or generate insoluble reactants, byproducts and/or additives. Water vapor selective materials also are advantageous because their use eliminates the need to use any further materials to retain soluble species in the apparatus, which also introduces additional steps and expense in the construction of the apparatus.

Preferably, the water vapor selective material has a thickness between about 2 microns and about 2,000 microns thick (e.g., 750 microns), a pore size between about 0.005 microns and about 10 microns, and a water intrusion pressure between about 20 millibars and about 6,000 millibars. Preferably, the water vapor selective material is adhered to or otherwise supported by a support layer that allows liquid water to permeate the support layer. The overall thickness of both the water vapor selective material and the support layer is preferably between about 1 mil and 20 mils. More preferred are water vapor selective materials having a thickness between about 15 microns and about 300 microns thick, a pore size between about 0.01 microns and about 5 microns, and a water intrusion pressure between about 100 millibars and about 2,500 millibars. Preferably, this layer has a water permeable support layer such that the total thickness of both layers is between about 2 mils and about 10 mils. Most preferred are water vapor selective materials having a thickness between about 20 microns and about 200 microns thick, a pore size between about 0.1 micron and about 3 microns (e.g., 0.5 microns), and a water intrusion pressure between about 200 millibars and about 1000 millibars. Preferably, this layer has a water permeable support layer such that the total thickness of both layers is between about 3 mils and about 8 mils.

Generally, if rapid generation and release of gas is desired, a thinner layer and/or large porosity is preferred. The thinner and/or more porous the layer, the more rapidly water vapor can diffuse into the apparatus and initiate the reaction, and the more rapidly gas can diffuse out of the apparatus. Lower porosity generally will result in slower gas generation and higher porosity generally will result in faster gas generation. If slower generation and release of gas is desired, a thicker material is preferred, such as between about 5 mils and about 20 mils. Additionally or alternatively, if slower generation and release of gas is desired, a relatively smaller surface area of the material can be used. For example, a sachet or a reactant chamber can be constructed from a rigid frame constructed from impermeable materials with water vapor selective materials sealing off one or more openings in the rigid frame.

Additionally or alternatively, pore size of the water vapor selective material can be selected to produce the desired release of gas at specific depths of water in which the apparatus will be used. A smaller pore size will correspond to deeper operation by increasing the amount of hydraulic water pressure that the membrane will experience while remaining impermeable to liquid water.

Water vapor selective materials suitable for use in constructing the apparatus of the present invention preferably have water vapor permeability of between about 500 g/m²/24 hrs and about 200,000 g/m²/24 hrs (e.g., 2,000 g/m²/24 hrs), as determined by JIS L 1099-1985 (Method B), “Testing Methods for Water Vapour Permeability of Clothes,” from the Japanese Standards Association. Water vapor selective materials preferably have a resistance to liquid water permeation of at least about 20 millibars as determined by ISO 811-1981 “Textile fabrics—Determination of resistance to water penetration—Hydrostatic pressure test” published by the International Organization for Standardization.

Optionally, the water vapor selective material(s) of the present invention can include a support layer to increase the strength of the layer, and/or to increase its ability to bond to the other materials used to construct the apparatus. The support layer preferably allows diffusion or passage of initiating agent to the surface of the water vapor selective material. For example, the support layer can be spun, perforated or have large pores that allow passage of liquid water and vapor to the surface of the water vapor selective material. The support layer can be affixed to the water vapor selective material by any means, e.g., lamination, casting, co-extrusion, and/or adhesive layers. The water vapor selective material can face the interior or exterior of the sachet and/or reactant chamber. The support layer itself can be hydrophilic and/or hydrophobic. If hydrophilic, the support layer can be used to attract and deliver liquid water and/or vapor to the surface of the water vapor selective material. Suitable support layers include, but are not limited to, polyethylene, polypropylene, nylon, acrylic, fiberglass, and polyester in the form of woven, non-woven, and mesh layers. Preferably, the support layer thickness is between about 1 mil and 25 mils.

Suitable water vapor selective materials include the 0.60 pore size, hydrophobic, polypropylene (PP) membrane having a thickness between about 250 microns and about 300 microns sold under the designation 060P1 by Cuno Incorporated (Meriden, Conn.). Also suitable is the 0.65 micron pore size, hydrophobic polyethylene material sold under the trade designation DOHP by Millipore (Bedford, Mass.). Another water vapor selective material, suitable for use in a rapid release apparatus, includes a 1.5 mil thick, hydrophobic polytetrafluoroethylene (PTFE) layer thermally bonded to a 5 mil thick, hydrophobic polyethylene (PE) support layer sold under the trade designation BHA-TEX® by BHA Technologies (Kansas City, Mo.). Its resistance to liquid water permeation is at least about 500 millibar. Also suitable are the polypropylene (PP) materials from Membrana GmbH (Wuppertal, Germany), sold under the trade names ACCUREL® PP2E HF (about 160 microns thick) and ACCUREL® PP1E (about 93 microns thick). These materials do not have a support layer, but one could be attached, if desired.

Alternatively or additionally, the gas permeable membrane includes a perforated material. Perforated materials suitable for use in accordance with the present invention include, but are not limited to, perforated materials having a water vapor transmission rate (WVTR) between about 50 g/m²/24 hrs and about 1,000 g/m²/24 hrs, between about 200 g/m²/24 hrs and about 800 g/m²/24 hrs, or between about 400 g/m²/24 hrs and about 700 g/m²/24 hrs. The measurement of water vapor transmission rate is routine and well known in the art. In some embodiments, the perforated material is hydrophobic, in others it is hydrophilic.

Exemplary perforated materials include Cryovac® polymeric perforated films available from Sealed Air Corporation (Duncan, S.C.). One such material is a hydrophobic polypropylene copolymer film sold under the designation SM700 by Sealed Air Corporation that has 330 holes per square inch having a diameter of 0.4 mm, a 6.4% perforated area, a thickness of about 20 microns, and a water vapor transmission rate of 700 g/m²/24 hrs. Another suitable film is a hydrophobic polypropylene copolymer material sold under the designation SM60 by Sealed Air Corporation and has 8 holes per square inch having a diameter of 0.4 mm, a 0.2% perforated area and a water vapor transmission rate of 65 g/m²/24 hrs. The artisan can readily identify suitable equivalents of any of the foregoing by exercising routine experimentation.

Alternatively or additionally, the gas permeable membrane for use in accordance with the present invention includes hydrophobic, liquid water permeable materials, such as polyethylene or polypropylene. These materials preferably are between about 1 mil and about 10 mils thick with a water intrusion pressure of about 20 millibars or less. Hydrophobic materials suitable for use as gas permeable materials in accordance with the present invention include, but are not limited to, non-woven polyethylene such as the TYVEK® non-woven polyethylenes from DuPont Company (Wilmington, Del.), for example, the TYVEK® 1025D non-woven polyethylene which has an intrusion pressure of less than 20 millibars.

Also suitable for use as gas permeable materials are membranes. Hydrophilic membranes, for example, a pore size between about 0.001 microns and about 50 microns, preferably between about 0.05 microns and 40 microns, and, more preferably, between about 0.1 and about 30 microns. Suitable membranes also include, but are not limited to, the microporous ultra high density polyethylene membrane sold under the trade designation MPLC from Millipore (Bedford, Mass.), and the microporous Nylon 6,6 membrane sold under the designation 045ZY by Cuno Incorporated (Meriden, Conn.).

Suitable extruded membranes, which include cast membranes, include 0.65 micron pore size, 230 to 260 micron thick, hydrophilic polyethylene membrane sold under the trade designation MPLC from Millipore (Bedford, Mass.), and 0.65 micron pore size, extruded hydrophobic polyethylene material sold under the trade designation DOHP by Millipore (Bedford, Mass.). Also suitable is the cast membrane 3 micron pore Nylon 6,6 material sold under the trade designation BIODYNE A by Pall (Port Washington, N.Y.). Suitable cast membranes include 0.45 pore, hydrophilic Nylon 6,6 membranes with a polypropylene backbone sold under the designation BA05 by Cuno Incorporated (Meriden, Conn.); 0.45 pore, hydrophilic polypropylene membrane available from 3M (St. Paul, Minn.); and 0.45 pore size, 180 to 240 micron thick, hydrophilic Nylon 6,6 membranes sold under the designations 045ZY and 045ZN by Cuno Incorporated (Meriden, Conn.). Also suitable are hydrophobic, liquid water permeable non-woven polyethylenes, such as the TYVEK® 1025D polyethylene material from DuPont Company (Wilmington, Del.).

Non-woven membranes can be formed, e.g., by suspending the membrane material, e.g., cellulose fibers, in a liquid over a porous web and then draining the liquid to form a membrane. Non-woven membranes typically have a relatively narrower and more consistent pore size distribution as compared to woven materials. Consequently, the material generally allows less initiating agent into the reactant chamber and/or sachet than a woven material having the same pore size. A non-woven membrane suitable for use in accordance with the present invention is the 0.65 micron pore size, hydrophobic, non-woven polypropylene material sold under the trade designation AN06 by Millipore (Bedford, Mass.).

Pore size affects the rate at which water and ions can diffuse through the material in both directions. In certain embodiments, the pore size of the material employed can be chosen to allow entry of initiating agent through the material and, at the same time, retain the reactants within the reactant chamber and/or sachet at a high concentration so that the reaction rate is increased and a high efficiency is maintained. Suitable membranes include those having a pore size between about 0.001 μm and about 50 μm, between about 0.05 μm and about 40 μm, or between about 0.10 μm and 30 μm.

The pore size of the materials also can be measured by bubble point. Bubble point is a measurement well known in the art which approximates pore size from a measurement of the pressure necessary to drive a bubble of gas through a wetted membrane. Suitable membranes include those having a bubble point between about 2 psi and about 100 psi, between about 5 psi and about 80 psi, or between about 10 psi and about 70 psi.

Suitable membranes also include those having a thickness between about 5 microns and 2,000 microns, more preferably between about 20 microns and 400 microns, and most preferably between about 75 microns and 300 microns.

Suitable membranes also include restricted access membranes which include membranes that are liquid water impermeable but restrict the passage of water ions and/or solubilized species such as solubilized reactants or byproducts.

In certain embodiments, the gas permeable membrane includes selective transmission materials. Selective transmission materials are neither perforated nor porous, but instead transfer gases through the polymer structure of the film. Selective transmission materials are multilayered or mixed polymer materials, where the layers and the polymers are chosen for controlled transmission of gases such as carbon dioxide and oxygen. Selective transmission materials are preferred in dry applications because it allows the gas to diffuse out of the reactant chamber or sachet, while retaining the initiating agent once released from a frangible pouch. Moreover, the selective transmission material increases the stability of the apparatus prior to its use because it does not easily allow the ingress of ambient water, which could prematurely initiate the reactants.

Generally, a material that has a high carbon dioxide transmission rate is preferred. While not wishing to be bound to any theory, it is believed that the carbon dioxide transmission rate approximates the chlorine dioxide transmission rate because chlorine dioxide and carbon dioxide are about the same size. Preferably, the selective transmission material has a selective gas transmission rate of between about 500 cc/m²/24 hrs and about 30,000 cc/m²/24 hrs for CO₂ and between about 1,000 cc/m²/24 hrs and about 10,000 cc/m²/24 hrs for O₂. More preferably, the material has a selective gas transmission rate of between about 1,000 cc/m²/24 hrs and about 25,000 cc/m²/24 hrs for CO₂ and between about 2,000 cc/m²/24 hrs and about 10,000 cc/M²/24 hrs for O₂. Most preferably, the material has a selective gas transmission rate of between about 5,000 cc/m²/24 hrs and about 25,000 cc/m²/24 hrs for CO₂ and between about 3,000 cc/m²/24 hrs and about 10,000 cc/m²/24 hrs for O₂. Measurement of selective gas transmission rate is routine and well known in the art. One suitable selective transmission material is a multilayered polymer film having a carbon dioxide transmission rate of 21,000 cc/m²/24 hrs and an oxygen transmission rate of 7,000 cc/m²/24 hrs sold under the trade designation PD-961 Cryovac® selective transmission film from Sealed Air Corporation (Duncan, S.C.).

Also suitable for use in the present invention as gas permeable membranes are woven materials such as materials woven from cotton, metal, polymer threads, metal threads or the like into a cloth or mesh.

The gas permeable materials of the invention can be constructed employing a material that is hydrophobic and/or hydrophilic. It can include, e.g., a material having one or more hydrophilic zones and one or more hydrophobic zones. These zones can be created, e.g., by chemically attaching, adhering and/or printing a functional chemical group or polymer onto a surface of the material that is hydrophilic, hydrophobic or charged to create one or more hydrophilic, hydrophobic or charged zones. For example, a sulfonic acid group can be disposed on the surface of the polypropylene membrane, creating zones that are both hydrophilic and negatively charged (R—SO₂⁻). The membrane can then be washed with a dilute acid such that the ion exchange groups (R—SO₂⁻) bind the H⁺ ions. These H⁺ ions can later be released to supply H⁺ ions to acid activate reactant, e.g., chlorite, as a replacement or supplement to acid reactant.

Also suitable are materials that have a first surface that is hydrophilic and a second surface that is hydrophobic. For example, such a material can have a hydrophilic surface facing the outside of the reactant chamber and a hydrophobic surface facing the interior of the reactant chamber. The exterior, hydrophilic surface can aid the initiation of the reaction since water will tend to wet the hydrophilic surface. The hydrophobic, interior surface limits water passage out of the volume. This keeps the reactants concentrated while allowing the gas to escape thereby exploiting the advantages of the discoveries disclosed herein. One such material suitable for use in the present invention is a non-woven membrane with a 0.65 micron pore size diameter formed from a hydrophobic material, such as polypropylene, that has been chemically functionalized with amines and carboxyl groups to produce a charged, hydrophilic surface.

Moreover, the gas permeable membrane may include composite materials, including, but not limited to, starch/polymer composite materials. Suitable composite materials include hydrophilic, 114 μm thick, non-woven rice starch/polyethylene composites sold under the designation 60MDP-P by Mishima Paper Company, Limited (Japan). This material is heat sealable and wets easily. Furthermore, this material can be used to construct compartment barriers and/or sachets that keep reactants separated until initiation, and/or can be used to control the rate of diffusion of reactants, the rate of gas diffusion, and the initiation of the reactant so that the reactant remains concentrated within the reactant chamber and the reaction is driven to completion.

Reactant Chamber

The reactant chambers, for use in the present invention, are defined at least in part by a gas permeable material and a resealable opening. Significantly, the reactant chamber is not necessarily formed entirely of the gas permeable membrane. In one embodiment, the reactant chamber is formed only in part, of gas permeable membranes, for example, in defined regions of permeability. Alternatively, the reactant chamber may be entirely formed of a gas permeable membrane.

The reactant chambers or sachets can include a barrier material and a gas permeable material sealed to each other to define a closed receptacle for reactant. Exemplary embodiments of such apparatus are depicted in the figures. Another example of such a reactant chamber or sachet is a volume defined by a rigid frame defining one or more openings and one or more gas permeable materials disposed about the one or more openings to define a closed receptacle for reactant. Further examples and embodiments are described in greater detail herein, and in the publications incorporated herein by reference.

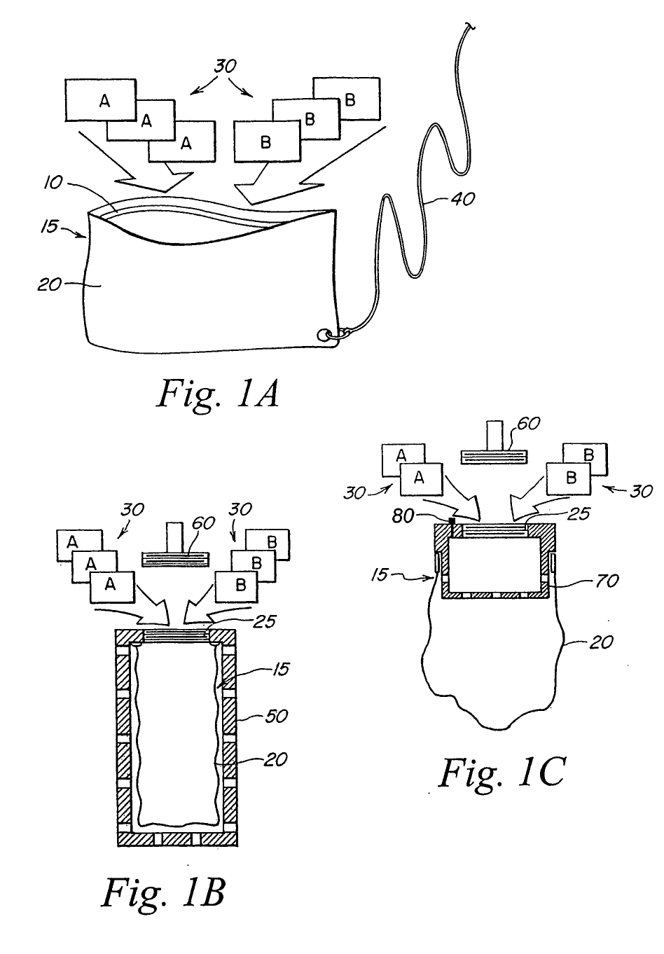

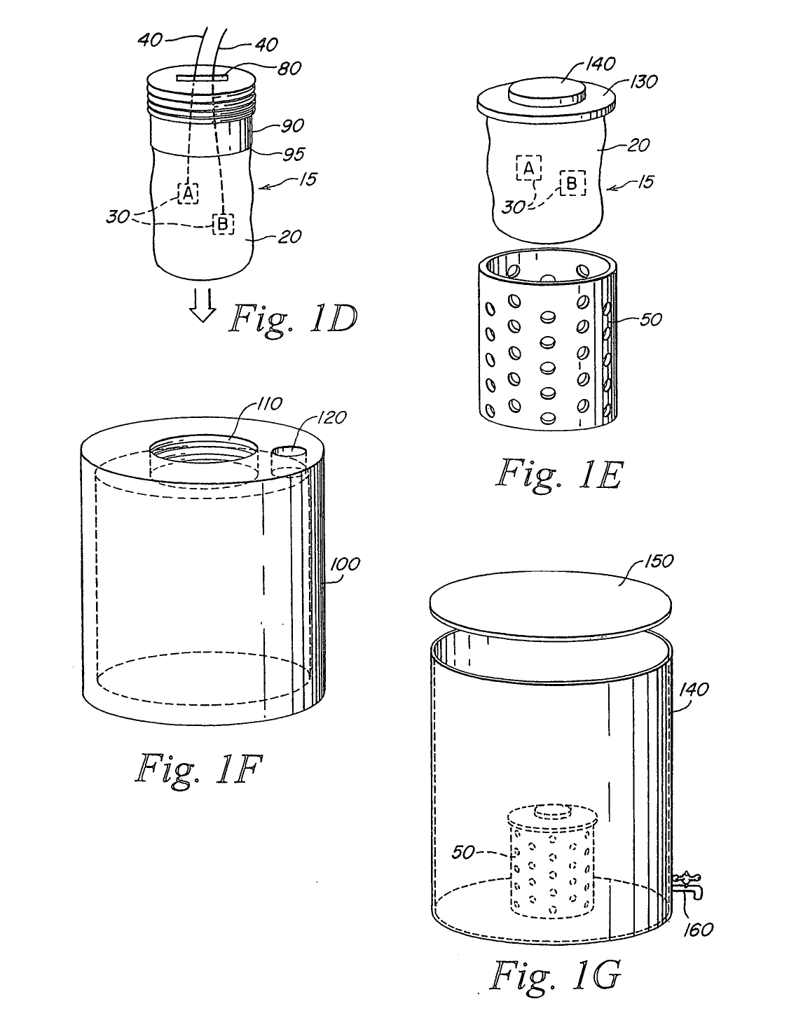

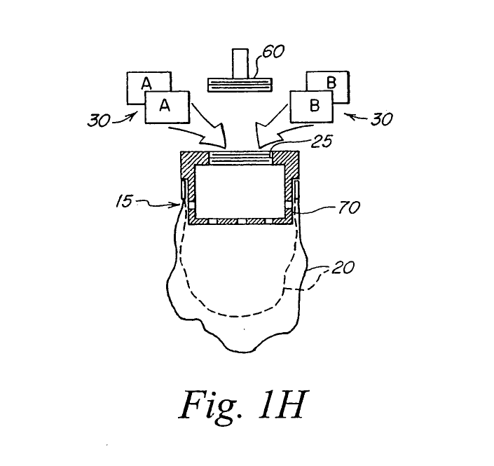

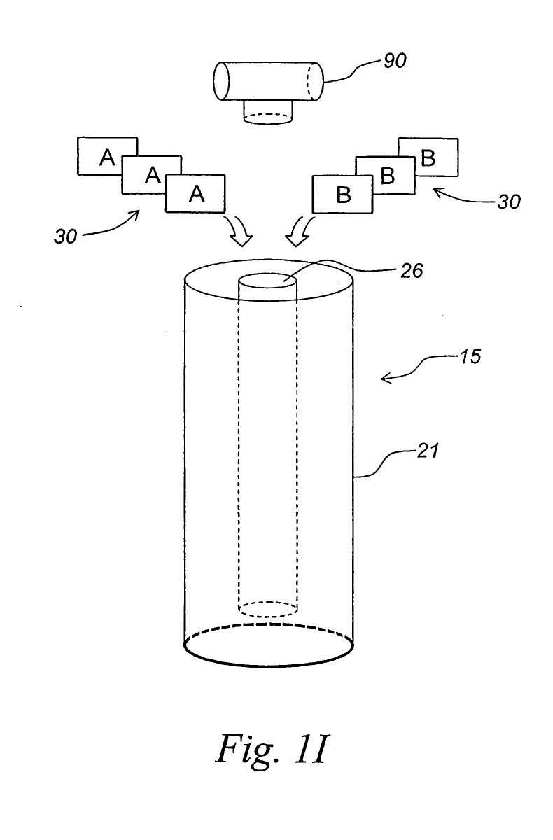

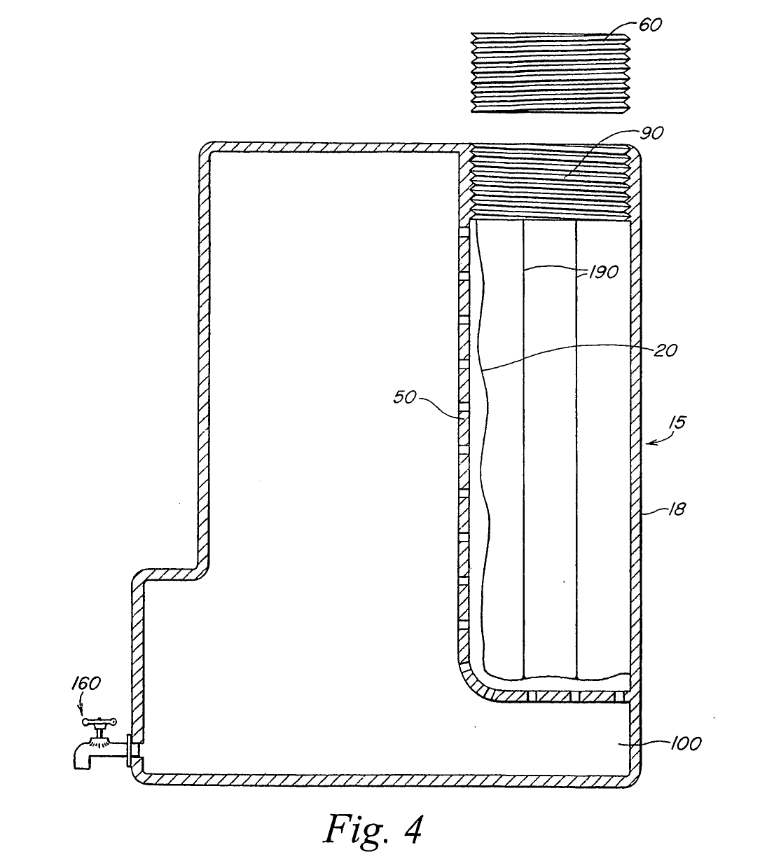

The ratio of reactant chamber volume to reactant volume also can be manipulated to control the concentration of the reactants, intermediates, byproducts, etc., within the reactant chamber. Increasing the concentration of reactants generally increases reaction efficiency. Preferably, the reactant chamber volume is less than about 20 times, 10 times, 6 times, 3 times or 2 times the volume of the reactant. In one embodiment, there is no head space such that the ratio is about 1. Smaller ratios are preferred in certain applications. For example, at small ratios, when the reaction produces water, the water increases the pressure inside the volume which, in turn, reduces the rate at which water can diffuse into the reactant chamber. The water to reactant ratio remains constant, thereby allowing the rate of reaction to remain constant. Any method of controlling, fixing or minimizing the void space may be employed in the present invention. By way of example, FIG. 1D and FIG. 4 show a “plug” device that facilitates minimizing the void space enclosed within the reactant chamber.

The reactant chamber can include more than one compartment. These sub-compartments of volumes can be defined at least in part by gas permeable and/or impermeable materials. These materials may be used to separate reactants as desired to facilitate the reaction, improve or retard the reaction speed, prevent premature initiation of the reaction or otherwise used. For example, a hydrophilic material and/or a highly permeable or porous material can be used to facilitate reaction at the interface between two compartments each containing a different reaction, e.g., citric acid and chlorite.

In certain embodiments, the reactant chamber may include suitable impermeable or barrier materials including, but not limited to, metals, glass, polymeric materials and/or coated papers. Other suitable materials include polymeric layers constructed from, e.g., polyethylene, polypropylene, polyester, styrene, such as polystyrene, polyethylene terephthalate, polyethylene terephthalate glycol (PETG), polyvinyl chloride, polyvinylidene chloride, ethylvinyl alcohol, polyvinyl alcohol, such as polyvinyl alcohol acetate, acrylobutylstyrene and/or polytetrafluoroethylene, polyacrylate and polyamide, such as nylon. Also suitable are metallized layers, e.g., any of the above polymeric layers that have been metallized. Also suitable are metallic foils, such as aluminum foils. Various other impermeable materials can be used to form the barrier film as well, such as glass or ceramics.

In addition, layers that are composites of the above layers and/or laminates of the above layers, e.g., paper/film/foil composites are also suitable. One such material is a 5 mil thick impermeable layer comprising a polyester exterior, a metallized biaxially oriented core, and a polyethylene interior sealing layer available from Sealed Air Corporation (Duncan, S.C.). This layer has a water vapor transmission rate of 0.01 g/100 in²/24 hrs at 70% relative humidity and 122° F. Another suitable impermeable material is constructed from polyvinyl chloride. Yet another exemplary material is constructed from polyethylene terephthalate glycol (PETG).

The geometry and size of the impermeable materials can be adapted to suit various parameters, including the amount and type of reactant to be used, the desired surface area of the gas permeable materials, and attachments for the storage and use of the apparatus. For example, the barrier material can be used to form a resealable opening and, optionally, additional portions of a reactant chamber (e.g., a barrier layer can form a cavity to receive reactant in a sachet).

The impermeable materials can be formed into various geometries by various manufacturing methods known in the art, including, but not limited to, horizontal form film seal, vertical form fill seal, blister pack, skin pack, injection molding, blow molding, thermoforming, cold forming fill seal, or mechanical forming. Of course, the barrier material also may be flexible and not formed into any particular geometry, but sealed about its perimeter to the gas permeable material and other elements of the apparatus.

Resealable Openings

The reactant chambers of the present invention are defined, in part, by a resealable opening. In certain embodiments, the resealable opening provides a means for removal and replacement of reactant, thereby allowing for reuse of the reactant chamber. Resealing of these volumes can be accomplished, e.g., by a simple adhesive or “zip-lock” mechanism, threaded lids, caps, clamps or similar devices. In one embodiment, the resealable opening is constructed from materials substantially impervious to solids, gels, liquids, and gases, at least for the time needed for the reactants to react and produce the gas. A resealable opening, when sealed can be airtight, but is not necessarily so. In one alternative embodiment, the resealable opening does not allow a gas generating device (e.g., reactant) to exit when sealed, but does allow exchange of liquid and gas.

Pressure Relief Valve and Techniques

Often, during practice of the invention, gas and/or hygroscopic pressure may accumulate in the reactant chamber. If this pressure distends the gas permeable material, some loss in efficiency or number of re-uses may result. Accordingly, in another embodiment of the present invention, a pressure relief valve or other device may be employed. FIG. 1C shows a relief device that allows escape of the gas and/or water from the reactant chamber. Alternatively or in addition, a pressure relieving technique (such as a momentary opening of the seal) may be employed.

Supporting Element

The reusable apparatus of the invention may further include a supporting element that can be disposed adjacent to at least a portion of the gas permeable material. The supporting element can optionally enclose the reactant chamber in its entirety. The supporting element may be used to facilitate the reaction by sinking or by fixation to specific locations within a vessel containing the initiating agent (water or water vapor in the case of chlorine dioxide). The supporting element may also afford some measure of protection of the reactant chamber, including the gas permeable material, and therefore extend the number of times these materials can be reused. The supporting element may have holes, perforations or slots. It may be hydrophilic and/or hydrophobic, and/or otherwise facilitate the controlled generation of the gas. The supporting element may also prevent excessive distention of the gas permeable material and thus improve efficiency or increase the number of re-uses.

Gas Generating Device

In one aspect, the present invention provides a gas generation device including a reservoir and the reusable apparatus disclosed herein. In a particular embodiment, the reservoir is adapted to receive at least a portion of one or more reactant chambers. The reusable apparatus can be affixed, permanently or removably to the device, or may be unaffixed to the device (e.g., the reusable apparatus may float within the reservoir). In another embodiment, the reactant chamber is in fluid communication with the reservoir, e.g., it can be disposed partially or fully within the reservoir. The reservoir can be, but is not limited to, a humidifier, a bottle, a spray bottle, a tank, a drum (e.g., a 55 gallon drum), a storage drum, a cartridge, an air conditioner reservoir, a conduit, and/or the like. The reservoir can be fitted with one or more devices for introduction and removal of the reservoir contents, such as a hose, conduit, spigot, valve or the like.

Reactants

In particular embodiments, the reusable apparatus includes one or more reactants disposed in the reactant chamber. Generally, the reactants may be for the generation of a particular type of gas, for example, chlorine dioxide. Aqueous solutions of a gas (chlorine dioxide, for example) may be generated in situ using methods and devices described in U.S. Pat. No. 6,602,466 of Hamilton et al., issued Aug. 5, 2003, U.S. Pat. No. 6,607,696 of Hamilton et al., issued Aug. 19, 2003, and U.S. Publication No. 20030053931A1 of Hamilton et al., published Mar. 20, 2003.

The reactants can be mixed or separated, e.g., by different reactant chamber compartments and/or sachets. For example, one reactant can be enclosed in a sachet, while the remaining reactants are loose in the reactant chamber. The sachet can include any of the gas permeable materials provided herein. In preferred embodiments, the reactant chamber, sachet, or both are constructed with water vapor selective materials. In a particular embodiment, the reactant(s) is incorporated in a sachet thay may be punctured in accordance with the invention, so as to release the reactants in the chamber. The reactants can be in the form of a tablet. In addition, the reactants can be in solid, liquid or gel form. In addition, the reactants can be in the form of a membrane shell having a wick, as described in International Publication No. WO02/00332. Initiating agent, e.g., water, can be provided in the reactant chamber. It can be added directly, with a reactant and/or in a frangible pouch that can be ruptured as desired. The reactants can be provided in the form of pellets, rings, disks and any other convenient form.

Reactants can be any of the reactants and/or reactant mixtures, including additives, described in U.S. Pat. No. 6,602,466 of Hamilton et al., issued Aug. 5, 2003, U.S. Pat. No. 6,607,696 of Hamilton et al., issued Aug. 19, 2003, and U.S. Publication No. 20030053931A1 of Hamilton et al., published Mar. 20, 2003.

The reactants and any additives (e.g., hydrotalcite) can be refreshed by opening the resealable opening, inserting fresh reactants and re-sealing the reactant chamber. The new or fresh reactants can be measured amounts of reactants that are provided (1) as separate or mixed reactants in bulk whereby the user measures the appropriate amounts of reactants, (2) as pre-measured packets properly packaged for a desired shelf life which are opened to dispense the measured amounts of reactants, (3) packets enclosed all or in part by barrier, hydrophobic, hydrophilic or dissolvable material or a combination thereof, either as mixed or separate reactants or (4) packets of a bifurcated enclosure comprised all or in part of barrier, hydrophobic, hydrophilic or dissolvable material or combination thereof with each enclosed volume containing one or more reactants. The figures show separated reactants as described above. However, any one or a combination of methods (1) through (4) above may be employed to introduce fresh reactants.

At times it may also be desirable to use several packets, as described in (3) and (4) above, of one or more of the reactants. Multiple packets can facilitate the initiation of the reaction, improve contact between reactants and alter the efficiency or speed of the reaction. It also allows the flexibility of allowing generation of desired amounts of gas and/or of providing different concentrations of gases in, for example, a reservoir, a chamber, or a room, as desired. For example, more reactant can be used to boost or initially clean a system, address a specific problem, and less reactant can thereafter be used to maintain a desired disinfection level. Instructions and/or charts can be provided that instruct the user how much reactant to add to achieve a desired level of gas and/or for a desired effect.

Generally, the reactions contemplated by the present invention include the reaction of oxyanions (e.g., carbonates, sulfates, sulfites, chlorates, chlorites, nitrites) and an acid in the presence of liquid water or water vapor. The exemplary oxyanions listed in the preceding sentence can also be generally categorized as non-metal oxides, which include the oxides of non-metals (carbon, sulfur, nitrogen, chlorine, etc.). Such non-metal oxides can be provided in salt form (e.g., with sodium or calcium as the cation) or in anionic form (e.g., in solution). Chlorine dioxide, e.g., can be generated by water activation of a mixture of sodium chlorite and an organic acid, such as citric acid. Gas generation can also be accomplished using an aqueous solution of either the acid or the sodium chlorite and initiating the reaction when the solution and the other dry ingredient mix. Other useful gases can also be generated by the initiation of a chemical reaction of dry chemicals by a fluid.

Generation of a gas, e.g., by acid activation, is well known in the art. For example, chlorine dioxide (ClO₂) is generated from sodium chlorite and an acid, such as citric acid. Alternatively, chlorine dioxide can be produced by the reduction of a chlorate, e.g., sodium chlorate or potassium chlorate, in the presence of an acid, e.g., oxalic acid. Another example of generation of a gas by acid activation is the activation of a sulfite, e.g., sodium bisulfite or potassium bisulfite, with an acid, e.g., fumaric acid and/or potassium bitartrate, in the presence of moisture to form sulfur dioxide. Yet another example is the acid activation of a carbonate, e.g., calcium carbonate with an acid, e.g., citric acid, to form carbon dioxide.

Other applications will be apparent to the skilled practitioner. For example, the generation of nitrogen dioxide by the acid activation of a nitrite, e.g., sodium nitrite or potassium nitrite. Alternative methods for gas generation, e.g., reduction of chlorates by sulfur dioxide (Mathieson Process), are well known in the art and also can be utilized in accordance with the present invention.

Any acid can be used as a reactant. However, weak acids are preferred, as they typically are safer to handle, produce less undesirable byproducts, and are less reactive. Also, multiprotic acids are preferred. Multiprotic acids are acids that have more than one reactive site. For example, the triprotic acid, citric acid, is preferred. Preferably, the aqueous soluble acid is selected from the group consisting of phosphoric acid, fumaric acid, glycolic acid, acetic acid, ascorbic acid, oxalic acid, maleic acid, lactic acid, tartaric acid, citric acid and mixtures thereof. More preferably, the aqueous soluble acid is selected from the group consisting of ascorbic acid, phosphoric acid, oxalic acid, maleic acid, lactic acid, tartaric acid, citric acid and mixtures thereof. Most preferably, the aqueous soluble acid is ascorbic acid, oxalic acid, citric acid and mixtures thereof.

It should be understood that the apparatus and methods of the present invention are readily applicable to the delivery of more than one gas at one time. For example, the reactant can include both a chlorite and a sulfite for the delivery of both chlorine dioxide and sulfur dioxide.

Preferably, the molar ratio of the non-metal oxide salt (e.g., sodium chlorite) to acid is between about 1:0.8 to about 1:5, preferably from about 1:1 to about 1:4, and most preferably from about 1:1.15 to about 1:3. The practitioner readily will appreciate that additional acid can be added to account for any additives or processing aids such as desiccants, stabilizers, flow agents and the like. Preferably, a pH between about 1.5 to about 5.5, more preferably a pH of about 3, is maintained by using an excess of acid. Because the reactants are concentrated within the reactant chamber, less acid is needed to drive the reaction to completion and the pH remains low because the acid is concentrated. Furthermore, the reactant salt is consumed by acid and therefore its presence upon completion of the reaction is minimized.

The reactants can be provided in a kit with one or more reusable apparatus and/or in refill kits for use with reusable apparatus. In the same vein, reusable reactant chambers and/or reusable apparatus of the invention can be provided with or without reactants and instructions. Kits can also include instructions for use, test strips for measuring gas concentration, charts that outline the amounts of reactant needed for various applications, and/or devices for measuring reactants. Kits can also include other additives, such as the stabilizers discussed below, flow agents, desiccants, and the like.

These additives can be included separately or can be incorporated into the reactant, e.g., in a pellet or a sachet.

According to the invention, hydrotalcite may be disposed within the chamber. Hydrotalcite, and the metal hydroxides in the hydrotalcite family, can be used to stabilize one or more reactants of the present invention. For example, hydrotalcite, including activated hydrotalcite, and metal hydroxides in the hydrotalcite family can be used to stabilize the following reactants and combination of these reactants: acids, aqueous acids, citric acid, oxalic acid, fumaric acid, phosphoric acid, potassium bitartate, chlorites, chlorates, sulfites, bisulfites, sulfates, carbonate, and nitrites. While not wishing to be bound to any particular theory, it is believed that the basic nature and ability of hydrotalcite and its family members to attract and bind water and water vapor contribute to their ability to stabilize the reactants of the present invention. Basic additives, compared to acidic additives, are less likely to react with chlorites and other reactants that react with acid. In addition, the ability of hydrotalcite and its family members to attract, absorb, adsorb and/or otherwise bind water and water vapor mitigates or prevents premature initiation, e.g., during manufacture, shipping, and storage. The ability of hydrotalcite to function as a flow agent also is desirable because it renders the reactants more readily metered and dispensed during manufacture. Thermally treated hydrotalcite, i.e., hydrotalcite that has been heated to drive off water and/or carbon dioxide, can be used to stabilize reactants in an apparatus, kit or refill kit such as those described in the instant application. Such apparatus are expected to have a shelf life of at least a year.

The mineral commonly known as hydrotalcite is the naturally occurring form of hydrotalcite, which has the chemical formula: Mg₆Al₂(OH)₁₆CO₃.4H₂O. Hydrotalcite, suitable for use in the apparatus of the present invention, is commercially available from DIP Chemical Industries (Mumbai, India). When thermally treated, this hydrotalcite is hygroscopic, in that it absorbs water vapor up to about 36% of its weight when exposed to about 97% relative humidity, and has a surface pH between about 11 and about 12. The hydrotalcite can be thermally treated using known methods, e.g., by exposing it to elevated temperatures for an extended period of time, e.g., 500° C. for one hour, and thereafter sealing the hydrotalcite in a low humidity environment (including the apparatus of the present invention) in order to minimize premature deactivation. Temperatures and times can be varied so as to allow, as desired, more or less water and/or carbon dioxide to be driven off. Such variations are within the scope of the invention. For example, the thermal treatment can be exposure to temperatures of 100° C., 200° C., 300° C., 375° C., etc. for 15 minutes, 30 minutes, 2 hours, etc.

There also are metal hydroxides in the hydrotalcite family that can be used to stabilize reactants in accordance with the present invention. The family can be described with the general formula: A_{w}Bₓ(OH)_{y}C_{z}.nH₂O, wherein A is a divalent metal cation, B is a trivalent metal cation, and C is a monovalent, divalent, trivalent, or tetravalent anion. The values for w, x, y, z, and n have the following relationship: 0≦z≦x≦4≦w≦1/2y, and 12≧n≧3/2x. Metal cations represented by A include, but are not limited to, Mg²⁺, Ni²⁺, Fe²⁺, and Zn²⁺. Metal cations represented by B include, but are not limited to, Al³⁺, Fe³⁺, and Cr³⁺. Chemical species represented by C include, but are not limited to CO₃²⁻, SO₄²⁻, OH⁻, NO₃⁻, and Cl⁻. One such hydrotalcite is a hydrotalcite having the formula: Mg₄Al₂(OH)₁₂CO₃.nH₂O, and is commercially available from Alcoa World Chemicals (Leetsdale, Pa.). Other suitable minerals in the hydrotalcite family include pyroaurite having the formula Mg₆Fe³⁺₂(OH)₁₆CO₃.4H₂O, and takovite having the formula Ni₆Al₂(OH)₁₆[(CO₃)_{0.75}(OH)_{0.25}].4H₂O.

Hydrotalcite can be added to the reactant such that the resulting mixture is between about 0.5% and about 40% hydrotalcite by weight. More preferably, the resulting mixture is between about 5% and about 20% hydrotalcite by weight.

Several embodiments are shown in the figures. The text hereafter will refer to devices that produce chlorine dioxide with water or water vapor as the initiating agent. One can extend the application of the devices to other gases and initiating agents.

FIGS. 1A-1C, 1H, and 1I depict kits including a plurality of reactant sachets and exemplary reusable apparatus of the present invention. FIG. 1A depicts a kit including a reactant chamber (15) defined by gas permeable material (20), and including a zipper or “zip-lock” resealable opening (10). Also shown are a plurality of reactants “A” and “B” (30) for insertion into the reactant chamber (15). Reactants (30) can be loose and/or disposed in sachets or other delivery devices. The reactant chamber (15) can be employed a plurality of times by virtue of its “zip-lock” resealable opening (10). The apparatus also features an optional tether (40) attached to the reaction chamber (15) for ease of retrieval from, e.g., a vessel.

FIGS. 1B and 1C feature kits having reactant chambers (15) defined in part by a gas permeable membrane (20), and having threaded resealabe openings (25) with lids (60) and supporting elements (50, 70) disposed adjacent the gas permeable material (20). Also shown are a plurality of reactants “A” and “B” (30) for insertion into the reactant chamber. Reactants (30) can be loose, compacted (e.g., pellets, disks or the like) and/or disposed within sachets. Supporting elements (50,70) can be employed to protect the membranes (20) either from external stresses or internal stresses. For example, in FIG. 1B, the supporting element (50) can protect the membrane (20) from puncture by foreign objects and/or can support the membrane (20) when under internal pressure during reaction. Similarly, in FIG. 1C, supporting element (70) can protect membrane (20) from reactants (30) and other materials (not shown) inserted into the reactant chamber. The supporting member can also be constructed from a material that allows the reactant chamber to sink or float in liquid, depending on the application. Furthermore, FIG. 1C features a pressure relief device (80).

FIGS. 1D-I are side views of exemplary reusable apparatus of the present invention, featuring a reactant chamber (FIGS. 1D-E and 1H-I), and a reservoir adapted to receive the reusable apparatus (FIGS. 1F-G).

FIG. 1D depicts a reactant chamber (15) including a gas permeable material (20), and a spring-loaded clamp resealable opening (80) that opens wide enough to accommodate the insertion or removal of reactants (30) that are attached to tethers (40) for ease of removal from the reactant chamber. The clamp (80) both secures the tethers and reseals the device. The use of the tethers is optional.

To maintain a fixed volume within the reactant chamber (15) and ensure that the reactants (30) are positioned in a manner that facilitates initiation of the reaction and/or exit of the gas thus generated into a specific place (e.g., below the “water line” of a container that will contain a chlorine dioxide solution), the threaded top (90) is configured to extend into reservoir (100) shown in FIG. 1F. This configuration ensures that the reactants remain submerged in any liquid in reservoir (100). Threaded top (90) can be threaded into mated threads (110) in reservoir (100) such that the bottom surface (95) of the threaded top (90) defines, in part, a sealed reservoir volume.

The reservoir (110) shown in FIG. 1F can be any container that holds water such as a 55-gallon drum, a 5-gallon pail or a custom-made container. Gas, such as chlorine dioxide, can applied using a pump, sprayer, hose or other device inserted into the hole (120) which can be sealed while the chlorine dioxide is generated, and opened to apply the chlorine dioxide solution.

FIG. 1E depicts a reusable apparatus including a reactant chamber (15) defined by a gas permeable material (20) and a lid (130) having a resealable opening (140). Reactants (30) can be placed in the reactant chamber as shown. The apparatus further includes a supporting element or cylindrical container (50) that is permeable to gases and liquids. The supporting element (50) can be constructed of material that supports and/or protects the gas permeable material (20), and/or causes the reactant chamber to sink when inserted into a reservoir (140) as shown in FIG. 1G. Reservoir (140) includes a lid (150), which can be employed during generation of gas and/or storage of gas solutions. Gas generated can be passed into solution if the reservoir (140) contains a liquid such as water. A removal device such as the spigot (160) can then be used to dispense the solution.

FIG. 1H depicts another exemplary reusable apparatus. The apparatus is similar to FIG. 1C, except that FIG. 1H features multiple gas permeable membranes (20).

FIG. 1I depicts a reusable apparatus having a reactant chamber (15) defined in part by a tubular membrane (21) made of a robust material and having relatively thick walls. The tubular membrane has at least one resealable opening (26) that can be reversibly sealed by a plug (90) or an alternative device, such as a clamp. Also shown are a plurality of reactants “A” and “B” (30) for insertion into the reactant chamber (15).

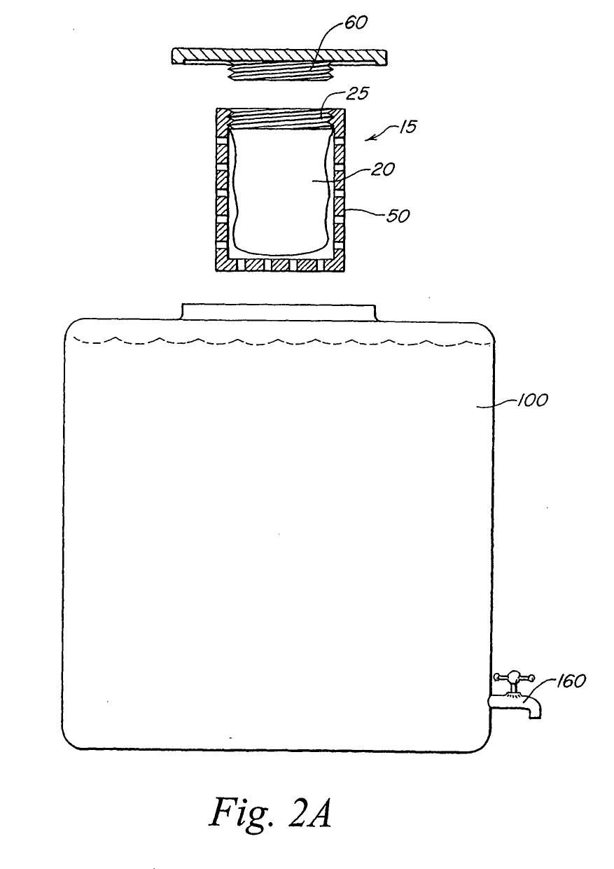

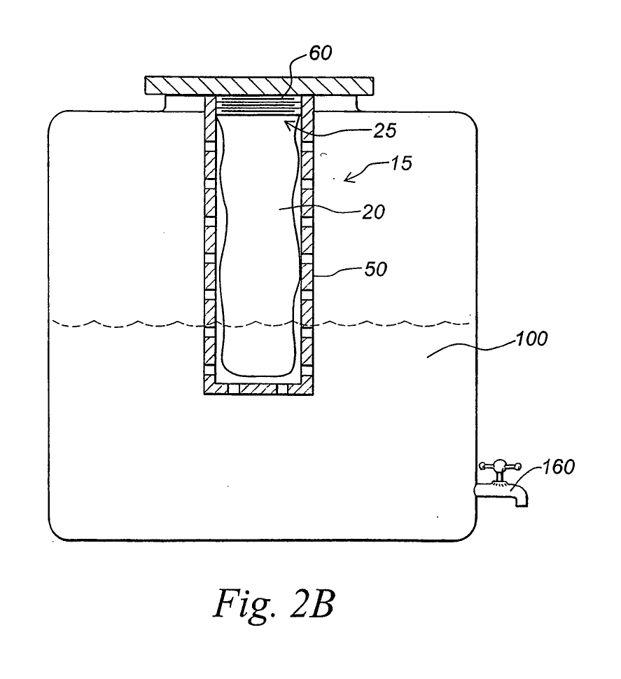

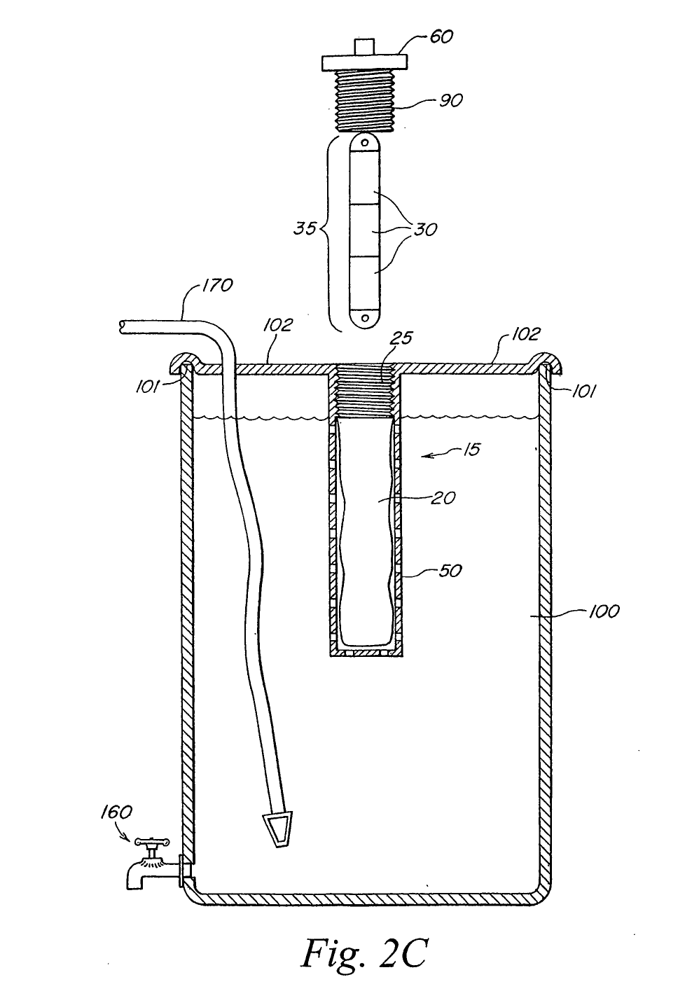

FIG. 2A is a side view of another exemplary reusable apparatus of the present invention featuring a reactant chamber (15) having a threaded resealable opening (25) including a mated lid (60) that is adapted to couple to a reservoir (100). Gas permeable material (20) is enclosed in a permeable supporting element (50) that is attached to the reactant chamber (15) about the resealable opening (25). Reactants (not shown) can be placed in the reactant chamber (15), and inserted into the reservoir (100), to submerge the reactants if sufficient liquid is provided. The resulting solution can be drawn off by means of a metering pump or spigot (160) as shown. Any alternative removal device can be used.

FIG. 2B is a side view of an exemplary apparatus of the invention (similar to that of FIG. 2A) showing the reactant chamber (15) partially submerged in liquid within a reservoir (100), thereby allowing gas to escape into both the liquid and the enclosed space above it.

FIG. 2C is a side view of another exemplary reusable apparatus of the present invention featuring a reactant chamber (15) having a threaded resealable opening (25) having a threaded insert (60) that also serves to seal the reservoir (100). The lid (102) attached to the resealable opening (25) extends out over the lip (101) of the reservoir (100). The reactants (30) can be refreshed by inserting an assembly of pre-packaged reactants (35) fixed to a holder (not shown) attached to a threaded insert (60). This combination of reactants and threaded insert (60) are screwed into the vessel or reservoir (100) in which the gas permeable material (20) is disposed. The threaded insert (60) has an extended portion (90) that ensures that the reactants are submerged when water fills the vessel (100). Also shown are an optional spigot (160) and hose (170). Either or both can be used to add and/or remove the contents of the reservoir (100). As shown, the reactant chamber is completely submerged in the liquid of the reservoir.

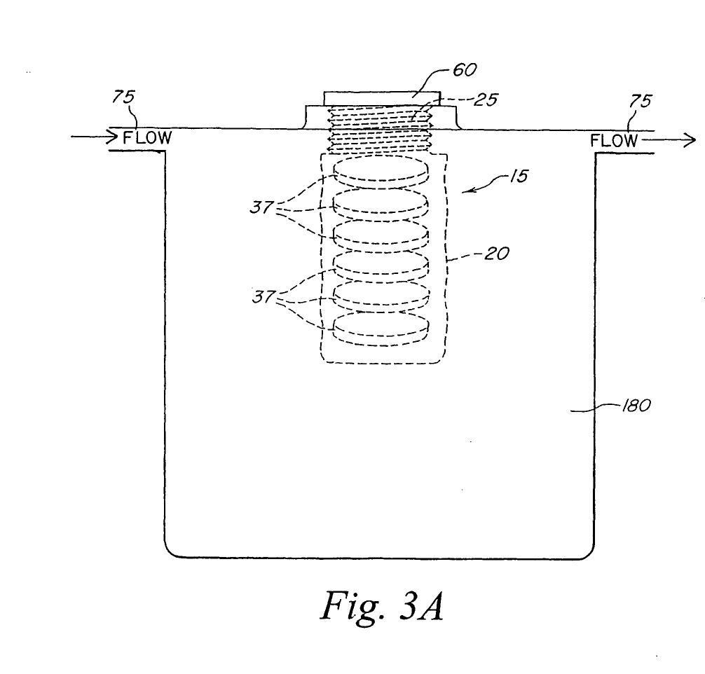

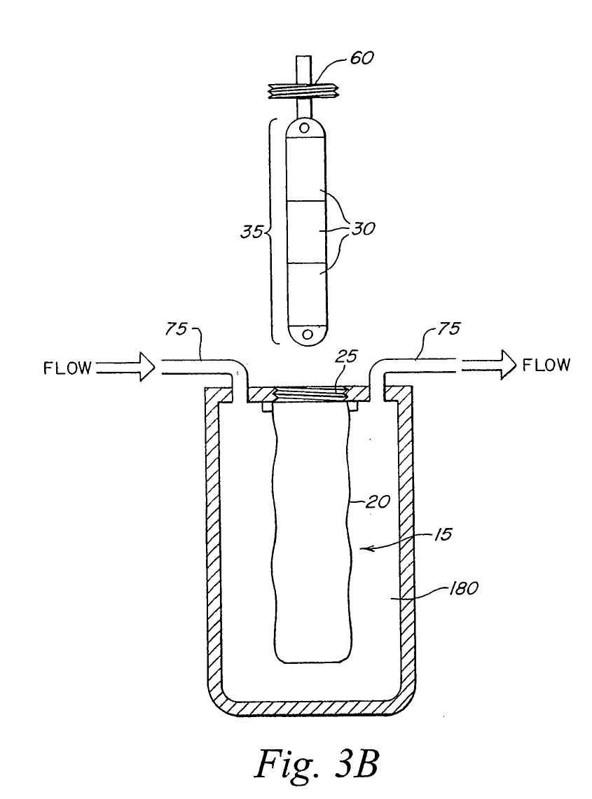

FIGS. 3A-B are side views of exemplary apparatus of the invention, each featuring a reusable reactant chamber (15), wherein the reactant chamber (15) is positioned in a reservoir (180) that is plumbed into a conduit (75). The reactant chamber (15) in each figure includes gas permeable material (20), and a resealable opening (25). The lid (60) not only defines the reactant chamber but also seals off the cartridge (180). The reactants (37, 30) can be refreshed via the resealable opening (25). Water or other fluids flow through conduit (75) and the cartridge (180) carrying the gas generated and released from the reactant chamber (15).

In FIG. 3A, the reactants are in the form of disks (37). The use of disks can be used to facilitate proper placement in the reactant chamber, allow variation in the amount of gas generated (by varying the number of disks inserted), improve contact between reactants, and minimize void space inside the reactant chamber. In FIG. 3B, the reactants (30) are contained in an assembly of pre-packaged reactants (35) fixed to a holder attached to lid or threaded insert (60).

FIG. 4 is a side view of an exemplary apparatus of the invention featuring a reactant chamber (15) defined by the gas permeable material (20), a side wall (18), and a resealable opening (25). The apparatus further includes a supporting element (50), a reservoir (100), and a spigot (160).

Gas permeable material (20) is affixed to the wall (18) of a larger vessel or reservoir (100). The boundary wall could be any other boundary wall of the reservoir. Resealable opening (25) includes a threaded lid (60) with a plug element (90) to maintain a specific volume within the reactant chamber, and to ensure that the reactants are completely submerged when the reservoir (100) is filled with a sufficient amount of liquid. Also shown are several “lanes” (190) defined by polymeric material (e.g., gas permeable, dissolvable, water permeable and/or impermeable materials) where reactants can be placed to at least initially maintain separation. Such separation can be used to enhance the generation of the chlorine dioxide or prevent reaction of the reactants prior to a desired time. Supporting element (50) can be used to support and/or protect the gas permeable material (20), and/or maintain a specific volume in the reactant chamber (15) as the supporting element (50) can impede the expansion of gas permeable material (20). The contents of the reservoir (100) can be drawn off by pump, sprayer, ladle or, as shown, via a spigot (160).

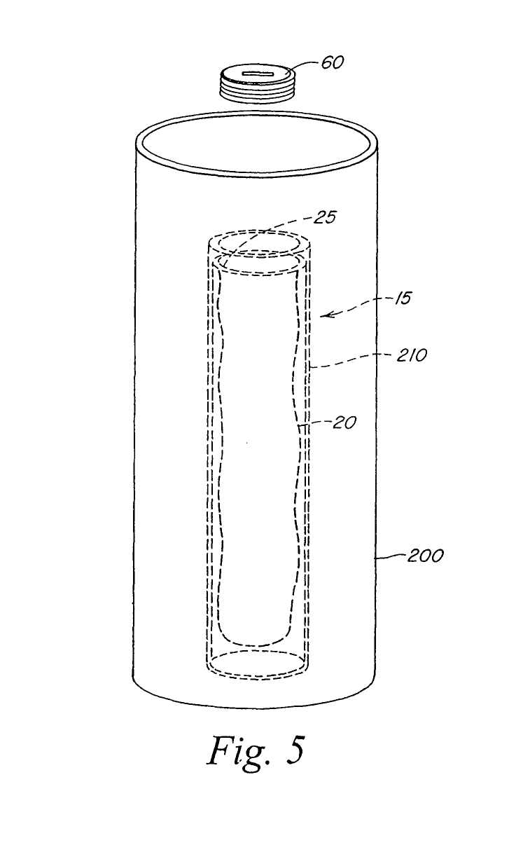

FIG. 5 is a side view of an exemplary apparatus of the invention featuring a reactant chamber (15) and a supporting element (210) adapted to receive it, which supporting element (210) can be introduced into or connected to a conduit (200). The reactant chamber (15) includes a gas permeable material (20), and a resealable opening (25) that includes a lid (60). Of course, the reactant chamber (15) and/or the supporting element (210) can be of any convenient shape that may be used to facilitate its use within a conduit and the conduit itself need not be circular in cross-section. The supporting element (210) can be placed or affixed into the conduit (200) and exposed to the initiating agent, e.g., water and/or water vapor. When the reactants (not shown) are exhausted, the reactants can be replaced in the reactant chamber (15) as needed. The supporting element (210) is optional, and can be used for a variety of purposes, e.g., to affix the reactant chamber (15) in the conduit (200), to protect the gas permeable material (20), and/or to confine the gas permeable material (20) to the boundary defined by supporting element (210).

The supporting element can be constricted from materials formed with open elements or orifices such as perforations, slots, grooves or the like. The supporting element can also have an open structure, such as an open weave or a spun structure. Optionally, the placement of the open elements of the supporting element can have directionality in that it controls which portion(s) of the supporting element allow the passage of gas and/or liquid. For example, slot can be defined in one area to direct the release of gas from the supporting element into its surrounding environment.

The foregoing figures depict exemplary embodiments illustrative of the invention, and are not meant to be exhaustive. The apparatus can be adapted for use in wet or dry applications, and each can readily be adapted to provide kits, reactant chambers, refill kits and reusable apparatus. For example, the apparatus of FIG. 2C can be provided with one or more reactants in a kit, or the apparatus can be sold without the reservoir with and without reactants. Further, the prepackaged reactants of FIG. 2C can be provided separately as refill kits. A variety of substitutions can be made that will be obvious to the skilled practitioner, e.g., the threaded resealable opening of FIG. 2C, could be replaced with a valve, clip, etc., and/or the lid attached to the reactant chamber could be otherwise adapted to fit the reservoir, and/or the reactants could be provided in alternative forms with or without additives, such as hydrotalcite, etc. A wide variety of gas permeable materials and sachet embodiments also can be used with the exemplary apparatus depicted in the figures.

The present invention can be used in a wide variety of applications. For example, chlorine dioxide can be used for the disinfection of water, e.g., water treatment; as a disinfectant for foods, beverages, fruits and vegetables; and for the cleaning and disinfection of medical, dental and food equipment. Chlorine dioxide has been shown to be an effective disinfectant at concentrations as low as 0.2 mg/l. Chlorine dioxide is a desirable replacement for chlorine, the traditional water treatment chemical, because it has been found to inactivate microbes at lower concentrations and over a wider pH range. For example, chlorine dioxide can be used to reduce or eliminate biofilms and/or free floating bacteria because it penetrates the cell wall of naturally occurring, colony-building microorganisms and disrupts the proteins necessary for reproduction. Moreover, chlorine dioxide does not produce chlorinated byproducts, e.g., trihalomethanes. Moreover, it has been found to be active against pathogens that are resistant to chlorine. It can be used as a slimicide in paper or pulp machines, for wastewater treatment, and for industrial water treatment, e.g., cooling or recycle streams. It can be used for odor control or as an aerial biocide and virucide. It can be used for the treatment of sulfides in the oil industry, for industrial cleaning, e.g., circuit board cleansing, and for paper or tallow bleaching. Sulfur dioxide also has a variety of uses, such as a mold and fungus inhibitor, for example, for use in shipping and storing fruits and vegetables. Based on the teachings disclosed herein, the practitioner of ordinary skill will appreciate the numerous other applications for which the present invention can be used and further provides the solution to a heretofore unmet need.

This invention is further illustrated by the following examples which should not be construed as limiting. The contents of all references, patents and published patent applications cited throughout this application, as well as the figures, are incorporated herein by reference.

### EXEMPLIFICATION

### EXAMPLE 1

### Construction and Re-Use of a Reusable Apparatus

Two reactant chambers were constructed, each 4.62 inches by 3 inches, and formed of Membrana ACCUREL® PP1E hydrophobic membrane sealed about three sides with one side open. Sachets of 3.5 inches by 2 inches, formed of Membrana polyethersulfone ACCUREL® Micro PES 6F hydrophilic membrane, were filled with 7.39 grams of dried citric acid monohydrate and sealed about their perimeter. A mixture of 95% technical sodium chlorite and 5% activated hydrotalcite was prepared.

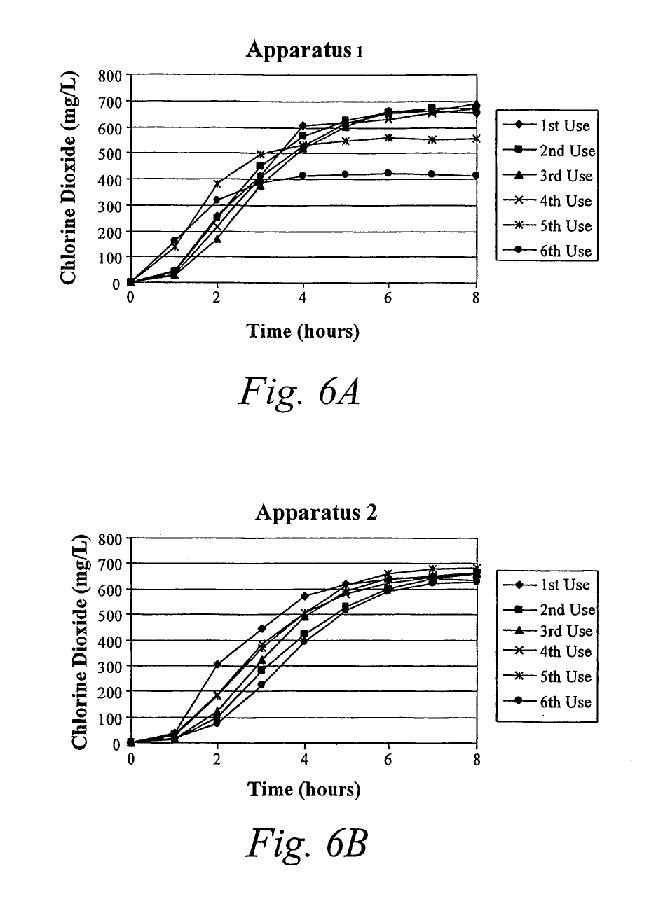

For each of six reuses of the reactant chamber, one sachet containing citric acid in addition to 4.95 grams of the mixture of technical sodium chlorite and activated hyrdotalcite were placed in the reactant chamber. The open side of the reactant chamber was closed with polyvinylchloride strips and metal clamps as submerged in two liters of water. The generation of chlorine dioxide, as measured by the concentration of the chlorine dioxide in solution in which the apparatus was submerged, is shown graphically in FIGS. 6A (Apparatus 1) and 6B (Apparatus 2).

### EXAMPLE 2

### Re-Use of a Reusable Apparatus Formed of Robust Material

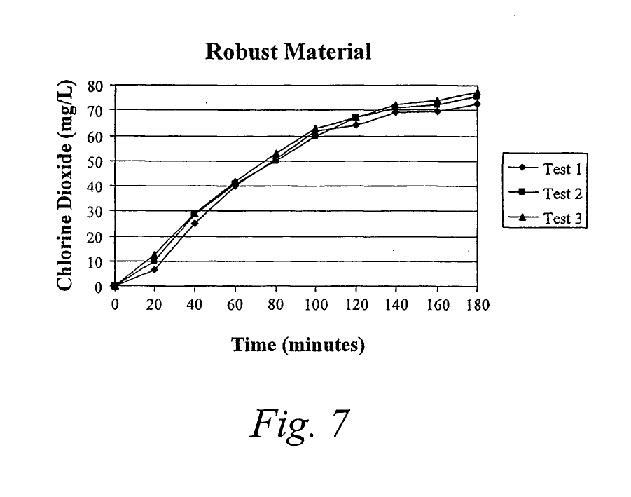

A device of Membrana ACCUREL® PP V8/2 BP hydrophobic tube membrane of 5.5 mm inner diameter with a wall of 1.55 mm thickness and 6.0 cm length was sealed at one end. The device was filled with 279 mg of dried citric acid monohydrate and 185 mg of a mixture consisting of 80% technical sodium chlorite and 20% activated hydrotalcite. The open end was sealed such that the length of the tube (not having the seals at both ends) was 4.5 cm long. The filled tube was immersed in 450 ml of water. The chorine dioxide generated was measured as chlorine dioxide concentration in the solution surrounding the device. The experiment was repeated three times. The chlorine dioxide generation is depicted in FIG. 7.

Equivalants

Although generally the preferred embodiments of the invention have been shown and described, numerous variations and alternative embodiments will occur to those skilled in the art. Accordingly, it is intended that the invention be limited only in terms of the appended claims as the invention can be embodied in other specific forms.

What is claimed is:

1. A reusable apparatus for the generation of a gas, the apparatus comprising:
a reactant chamber defined at least in part by a gas permeable material and a resealable opening.

2. The apparatus of claim 1, wherein the gas permeable material is substantially impervious to the passage of liquid.

3. The apparatus of claim 1, wherein the gas permeable material allows for the controlled passage of gas.

4. The apparatus of claim 1, wherein the reactant chamber further comprises a gas impermeable material.

5. The apparatus of claim 1, wherein the resealable opening is adapted for closure with a cap, a lid, a clamp, or a zip-lock mechanism.

6. The apparatus of claim 1, further comprising one or more reactants disposed in the chamber.

7. The apparatus of claim 6, wherein at least one of the reactants is disposed in a sachet or a frangible device.

8. The apparatus of claim 6, wherein a hydrotalcite is disposed in the chamber.

9. The apparatus of claim 8, wherein the hydrotalcite is thermally treated hydrotalcite.

10. A device for generation of gas comprising a reservoir and the reusable apparatus of claim 1.

11. The device of claim 10, wherein the reservoir is in fluid communication with the reactant chamber.

12. The device of claim 10, wherein the reservoir is selected from the group consisting of a humidifier, a spray bottle, a tank, a cartridge, a bottle, a drum, a storage drum, an air conditioner reservoir, and a conduit.

13. A method of generating gas, the method comprising the steps of:
providing a reusable apparatus comprising a reactant chamber defined at least in part by a gas permeable material and a resealable opening, wherein one or more reactants are disposed in the chamber; and
exposing the one or more reactants to an initiating agent, thereby generating gas.

14. The method of claim 13, wherein the gas comprises chlorine dioxide.

15. The method of claim 13, wherein at least one of the reactants is disposed in a frangible device or a sachet.

16. The method of claim 15, further comprising puncturing the frangible device.

17. The method of claim 13, wherein the initiating agent comprises water vapor, water or a combination thereof.

18. The method of claim 13, further comprising the step of puncturing a frangible device containing the initiating agent, thereby exposing the one or more reactants to the initiating agent.

19. The method of claim 13, further comprising the step of exposing the apparatus to an environment comprising the initiating agent, thereby exposing the one or more reactants to the initiating agent.

20. The method of claim 13, further comprising the step of partially or completely submerging the apparatus in the initiating agent.

21. The method of claim 13, wherein the gas permeable material is substantially impervious to the passage of liquid.

22. The method of claim 13, wherein the gas permeable material allows for the controlled passage of gas.

23. The method of claim 13, wherein the reactant chamber further comprises a gas impermeable material.

24. The method of claim 13, wherein the resealable opening is adapted for closure with a cap, a lid, a clamp, or a zip-lock mechanism.

25. The method of claim 13, further comprising providing a reservoir in fluid communication with the reactant chamber.

26. A kit comprising:
a reusable reactant chamber defined at least in part by a gas permeable material; and
one or more reactants.

27. A refill kit comprising one or more reactants for use in a reusable reactant chamber.

28. The refill kit of claim 27 comprising a means for delivery of the one or more reactants to a reusable reactant chamber.
